(19) United States
(12) Patent Application Publication
(10) Pub. No.: US 2009/0036368 A1
(43) Pub. Date: Feb. 5, 2009

(54) Therapeutic Combination

(75) Inventors: Patrick Brest, Nice (FR); Catharina Svanborg, Lund (SE)

Correspondence Address: NIXON & VANDERHYE, PC, 901 NORTH GLEBE ROAD, 11TH FLOOR, ARLINGTON, VA, 22203, US

(73) Assignee: HAMLET PHARMA AB, Lund (SE)

(21) Appl. No.: 11/991,926

(22) PCT Filed: Sep. 14, 2006
(86) PCT No.: PCT/IB2006/002535

§ 371 Date: Sep. 19, 2008

(30) Foreign Application Priority Data
Sep. 14, 2005 (GB) 0518720.8

(51) Int. Cl. A61K 38/17; A61P 35/00

(52) U.S. Cl. 514/12

(57) ABSTRACT

A combination of component (i) which is HAMLET or a biologically active modification thereof, or a biologically active fragment of either of these, and component (ii) which is a histone deacetylase (HDAC) inhibitor. This combination shows synergistic effects in the treatment of for example proliferative diseases such as those which produce tumours.

The present invention relates to combinations of biologically active components, which have been found to be particularly effective in the treatment of proliferative disease such as those which give rise to tumours such as cancer.

HAMLET (Human α-lactalbumin made lethal to tumour cells) is a molecular complex that induces cell death in tumour cells. Indeed, the effect is selective for tumour cells and some immature cells and healthy, differentiated cells do not undergo cell death in response to HAMLET. This selectivity implies that HAMLET reaches unique targets in tumour cells, but not in resistant cells.

Biologically active variants or derivatives of this complex with similar activity are described for example in International Patent Application No. PCT/IB03/01293.

The cellular targets for HAMLET have been examined by a combination of confocal microscopy and subcellular fractionation [Håkansson et al., 1999 Exp Cell Res. 246, 451-60]. HAMLET binds to the cell surface, and enters the cytoplasm where it interacts with and activates mitochondria. Finally, the protein enters the cell nuclei, where it accumulates.

The applicants have found that resistant and sensitive cells bind HAMLET to their surface with similar efficiency, suggesting that this is not the discriminating event. The nuclear accumulation, in contrast, occurs only in dying cells, suggesting that this step distinguishes sensitive from resistant cells. By confocal microscopy, the nuclear accumulation appeared irreversible, suggesting the presence of nuclear targets that bind and retain HAMLET in the nuclear compartment.

The applicants have conducted a closer examination of the intranuclear distribution of HAMLET and have found that the protein preferentially localized to areas corresponding to the nucleoli, implying that HAMLET interacts with molecules involved in the regulation of chromatin structure.

As a result of further study, the nuclear targets for HAMLET were identified. Surprisingly it was found that HAMLET interacts with specific histone proteins (see WO2003/098223), in a manner which is independent of the presence of the histone tail. It appears therefore that this interaction may be the event that irreversibly locks the cells into the death pathway. HAMLET appears to be unique in having this mode of action in the anti-tumour field.

However, nothing is known about its mechanism of action, and in particular, the mechanism of chromatin perturbation and the effects on cell viability are not known. Histone acetylases (HAT) and histone deacetylases (HDAC) regulate the acetylation and the deacetylation of the lysine residues in the histone tails, respectively. Increased histone acetylation attenuates the electrostatic interaction with the negatively charged bases, decreasing the interaction of the histones with DNA and allowing the access of transcription factors to target genes.

Histone deacetylases (HDACs) are involved in multiple cancers, as they repress the transcription of multiple genes, including tumour suppressor genes.

HDACs have emerged as molecular targets for the development of enzymatic inhibitors to treat human cancer. HDACs are generally over-expressed in tumours and promote tumour cell longevity by blocking transcription of anti-tumoral genes like p21WAF1 and P27KIP1. Many HDAC inhibitors are currently used in vivo and in vitro due to their activity against variety of human malignancies.

For example, the HDAC inhibitors trichostatin A (TSA) and suberoylanilide hydroxamic acid (SAHA) have been shown to have activities on breast cancer and prostate cancer respectively both in vivo and in vitro and depsipeptide was also recently shown to have clinical activities during the treatment of T cell lymphoma in early Phase I/II trials.

In addition, HDAC inhibitors have been used in combination with other anti-tumoral drugs in cancer therapy with varying degrees of success. When used with chemotherapeutic agents for example, combined effects ranged from being synergistic to being antagonistic or increasing toxicity.

Enhanced or synergistic effects were noted when HDAC inhibitors were combined with demethylating agents, nuclear receptor ligands, signal transduction inhibitors and Hsp90 antagonists and proteasome inhibitors (Drummond et al. supra).

The interaction whereby HDAC inhibitors promote other treatments is not completely understood. However, when dealing with a molecule with a completely different mode of action to any of the known therapeutics, such as HAMLET, it is not possible to predict how combination therapy with HDAC inhibitors would operate, particularly as both agents appear to interact directly with the same target, i.e. histone, albeit at different sites within the histone molecule.

The applicants have found however, that although HAMLET is not an HDAC inhibitor, HAMLET-induced cell death is enhanced in a synergistic manner when used in conjunction with HDAC inhibitors, giving rise to a new combined therapeutic approach.

According to the present invention there is provided a combination of component (i) which is HAMLET or a biologically active modification thereof, or a biologically active fragment of either of these, and component (ii) which is a histone deacetylase (HDAC) inhibitor.

As used herein, the term “HAMLET” refers to a biologically active complex of α-lactalbumin (which may or may not be human in origin), which is either obtainable by isolation from casein fractions of milk which have been precipitated at pH 4.6, by a combination of anion exchange and gel chromatography as described for example in EP-A-0776214, or by subjecting α-lactalbumin to ion exchange chromatography in the presence of a cofactor from human milk casein, characterized as C18:1 fatty acid as described in WO99/26979.

In order to form biologically active complexes, α-lactalbumin generally requires both a conformational or folding change as well as the presence of a lipid cofactor. The conformational change is suitably effected by removing calcium ions from α-lactalbumin. In a preferred embodiment, this is suitably facilitated using a variant of α-lactalbumin which does not have a functional calcium binding site.

Biologically active complexes which contain such variants are encompassed by the term “modifications” of HAMLET as used herein. However, the applicants have found that, once formed, the presence of a functional calcium binding site, and/or the presence of calcium, does not affect stability or the biological activity of the complex. Biologically active complexes have been found to retain affinity for calcium, without loss of activity. Therefore complex of the invention may further comprise calcium ions.

Thus in particular, the invention uses a biologically active complex comprising alpha-lactalbumin or a variant of alpha-lactalbumin which is in the apo folding state, or a fragment of either of any of these, and a cofactor which stabilises the complex in a biologically active form, provided that any fragment of alpha-lactalbumin or a variant thereof comprises a region corresponding to the region of α-lactalbumin which forms the interface between the α and β domains.

Suitably the cofactor is a cis C18:1:9 or C18:1:11 fatty acid or a different fatty acid with a similar configuration.

In a particular convenient embodiment, the biologically active complex used in the invention comprises

(i) a cis C18:1:9 or C18:1:11 fatty acid or a different fatty acid with a similar configuration; and
(ii) α-lactalbumin from which calcium ions have been removed, or a variant of α-lactalbumin from which calcium ions have been released or which does not have a functional calcium binding site; or a fragment of either of any of these, provided that any fragment comprises a region corresponding to the region of α-lactalbumin which forms the interface between the alpha and beta domains.

As used herein the expression “variant” refers to polypeptides or proteins which are homologous to the basic protein, which is suitably human or bovine α-lactalbumin, but which differ from the base sequence from which they are derived in that one or more amino acids within the sequence are substituted for other amino acids. Amino acid substitutions may be regarded as “conservative” where an amino acid is replaced with a different amino acid with broadly similar properties. Non-conservative substitutions are where amino acids are replaced with amino acids of a different type. Broadly speaking, fewer non-conservative substitutions will be possible without altering the biological activity of the polypeptide. Suitably variants will be at least 60% identical, preferably at least 70%, even more preferably 80% or 85% and, especially preferred are 90%, 95% or 98% or more identity.

Identity in this instance can be judged for example using the BLAST program or the algorithm of Lipman-Pearson, with Ktuple:2, gap penalty:4, Gap Length Penalty:12, standard PAM scoring matrix (Lipman, D. J. and Pearson, W. R., Rapid and Sensitive Protein Similarity Searches, Science, 1985, vol. 227, 1435-1441).

The term “fragment thereof” refers to any portion of the given amino acid sequence which will form a complex with the similar activity to complexes including the complete α-lactalbumin amino acid sequence. Fragments may comprise more than one portion from within the full length protein, joined together. Portions will suitably comprise at least 5 and preferably at least 10 consecutive amino acids from the basic sequence.

Suitable fragments will be deletion mutants suitably comprise at least 20 amino acids, and more preferably at least 100 amino acids in length. They include small regions from the protein or combinations of these.

The region which forms the interface between the alpha and beta domains is, in human α-lactalbumin, defined by amino acids 34-38 and 82-86 in the structure. Thus suitable fragments will include these regions, and preferably the entire region from amino acid 34-86 of the native protein.

In a particularly preferred embodiment, the biologically active complex comprises a variant of α-lactalbumin in which the calcium binding site has been modified so that the affinity for calcium is reduced, or it is no longer functional.

It has been found that in bovine α-lactalbumin, the calcium binding site is coordinated by the residues K79, D82, D84, D87 and D88. Thus modification of this site or its equivalent in non-bovine α-lactalbumin, for example by removing one of more of the acidic residues, can reduce the affinity of the site for calcium, or eliminate the function completely and mutants of this type are a preferred aspect of the invention.

The Ca²⁺-binding site of bovine α-lactalbumin consists of a 3₁₀ helix and an α-helix with a short turn region separating the two helices (Acharya K. R., et al., (1991) J Mol Biol 221, 571-581). It is flanked by two disulfide bridges making this part of the molecule fairly inflexible. Five of the seven oxygen groups that co-ordinate the Ca²⁺ are contributed by the side chain carboxylates of Asp82, 87 and 88 or carbonyl oxygen's of Lys79 and Asp84. Two water molecules supply the remaining two oxygen's (Acharya K. R., et al., (1991) J Mol Biol 221, 571-581).

Site directed mutagenesis of the aspartic acid at position 87 to alanine (D87A) has previously been shown to inactivate the strong calcium-binding site (Anderson P. J., et al., (1997) Biochemistry 36, 11648-11654) and the mutant proteins adopted the apo-conformation.

Therefore in a particular embodiment, the aspartic acid residue at amino acid position 87 within the bovine α-lactalbumin protein sequence is mutated to a non-acidic residue, and in particular a non-polar or uncharged polar side chain.

Non-polar side chains include alanine, glycine, valine, leucine, isoleucine, proline, phenylalanine, methionine, tryptophan or cysteine. A particularly preferred example is alanine.

Uncharged polar side chains include asparagine, glutamine, serine, threonine or tyrosine.

In order to minimize the structural distortion in the mutant protein, D87 has also been replaced by an asparagine (N) (Permyakov S. E., et al., (2001) Proteins Eng 14, 785-789), which lacks the non-compensated negative charge of a carboxylate group, but has the same side chain volume and geometry. The mutant protein (D87N) was shown to bind calcium with low affinity (K-_{Ca}2×10⁵M⁻¹) (Permyakov S. E., et al., (2001) Proteins Eng 14, 785-789). Such a mutant forms an element of the biologically active complex in a further preferred embodiment of the invention.

Thus particularly preferred variants for use in the complexes of the invention are D87A and D87N variants of α-lactalbumin, or fragments which include this mutation.

This region of the molecule differs between the bovine and the human proteins, in that one of the three basic amino acids (R70) is changed to S70 in bovine α-lactalbumin thus eliminating one co-ordinating side chain. It may be preferable therefore, that where the bovine α-lactalbumin is used in the complex of the invention, an S70R mutant is used.

The Ca²⁺ binding site is 100% conserved in α-lactalbumin from different species (Acharya K. R., et al., (1991) J Mol Biol 221, 571-581), illustrating the importance of this function for the protein. It is co-ordinated by five different amino acids and two water molecules. The side chain carboxylate of D87 together with D88 initially dock the calcium ion into the cation-binding region, and form internal hydrogen bonds that stabilise the structure (Anderson P. J., et al., (1997) Biochemistry 36, 11648-11654). A loss of either D87 or D88 has been shown to impair Ca2+ binding, and to render the molecule stable in the partially unfolded state (Anderson P. J., et al., (1997) Biochemistry 36, 11648-11654).

Further, mutant proteins with two different point mutations in the calcium-binding site of bovine α-lactalbumin may be used. For example, substitution of the aspartic acid at position 87 by an alanine (D87A) has been found to totally abolish calcium binding and disrupt the tertiary structure of the protein. Substitution of the aspartic acid by asparagine, the protein (D87N) still bound calcium but with lower affinity and showed a loss of tertiary structure, although not as pronounced as for the D87A mutant (Permyakov S. E., et al., (2001) Proteins Eng 14, 785-789). The mutant protein showed a minimal change in packing volume as both amino acids have the same average volume of 125 Å³, and the carboxylate side chain of asparagines allow the protein to co-ordinate calcium, but less efficiently (Permyakov S. E., et al., (2001) Proteins Eng 14, 785-789). Both mutant proteins were stable in the apo-conformation at physiologic temperatures but despite this conformational change they were biologically inactive. The results demonstrate that a conformational change to the apo-conformation alone is not sufficient to induce biological activity.

The structure of α-lactalbumin is known in the art, and the precise amino acid numbering of the residues referred to herein can be identified by reference to the structures shown for example in Anderson et al. supra. and Permyakov et al supra.

In a particularly preferred embodiment however, component (i) within the combination is HAMLET, obtainable from human milk.

As for component (ii), there are six main classes of know HDAC inhibitors and these can be summarised as follows:

A. HDAC inhibitory carboxylic acids of low molecular weight (for example of molecular weight less than 150) or pharmaceutically acceptable salts or pharmaceutically acceptable esters thereof, such as salts of butyric acid, for instance sodium butyrate, valproic acid or pharmaceutically acceptable salts or pharmaceutically acceptable esters thereof, phenyl butyric acid or pharmaceutically acceptable salts or pharmaceutically acceptable esters thereof, for instance sodium phenylbutyrate or pivalyloxymethylbutyrate (AN9).
B. Hydroxamic acids such as suberoylanilide hydroxamic acid (SAHA) of structure (i),

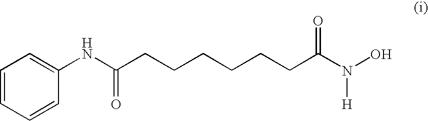

NVP-LAQ-824 of structure (ii)

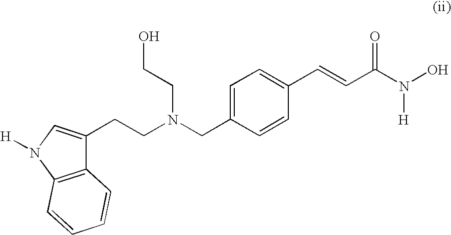

m-Carboxycinnamic acid bishydroxamic acid (CBHA) of structure (iii)

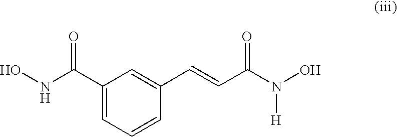

suberic bishydroxamic acid (SBHA) of structure (iv)

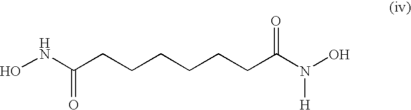

JNJ16241199 of structure (v)

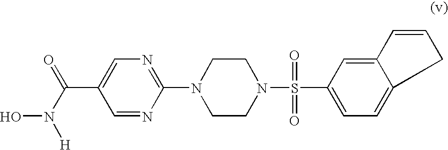

Proxamide of structure (vi)

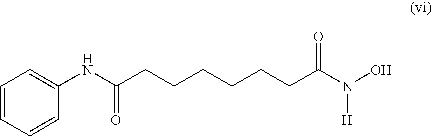

Scriptaid of structure (vii)

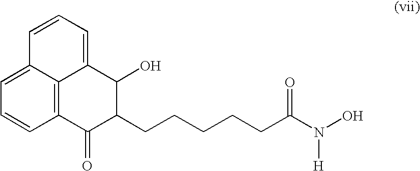

Oxamflatin of structure (viii)

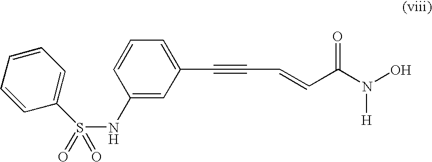

Trichostatin A (TSA) of structure (ix)

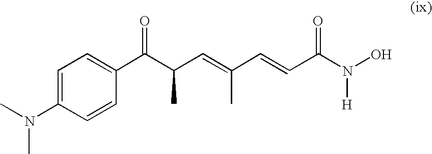

Tubacin of structure (x)

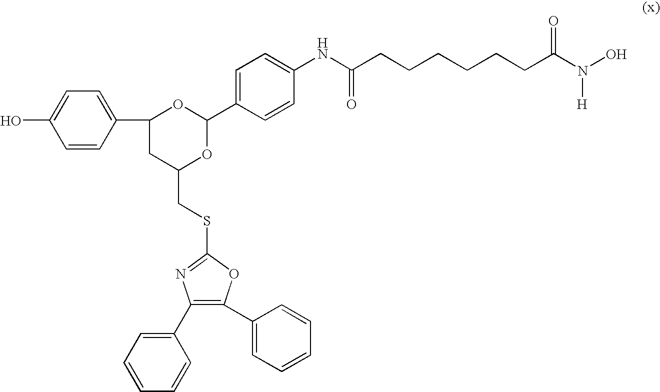

6-(3-Chlorophenylureido)caproic hydroxamic acid (3-Cl-UCHA) of structure (xi)

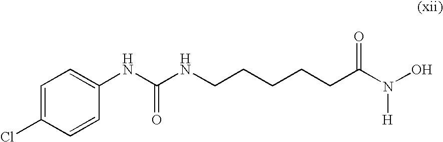

and A-161906 of structure (xiii)

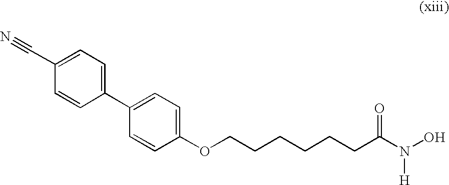

C. Benzamides such as MS-275 of structure (xiv)

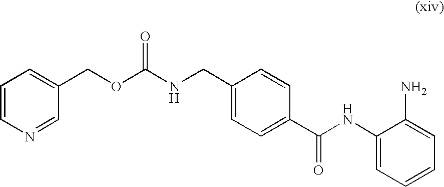

or N-acetyl dinaline (Cl-994) of structure (xv)

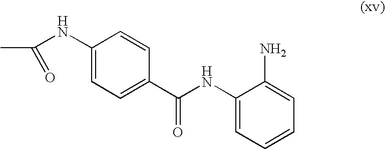

D. Epoxyketones such as 2-amino-8-oxo-9,10-epoxydecanoic acid (AOE) of structure (xvi)

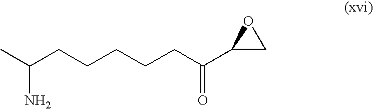

or Trapoxin (TPX) of structure (xvii)

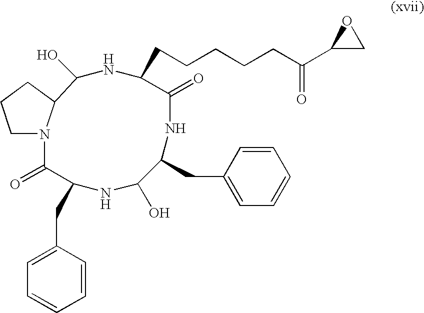

E. Cyclic peptides such a Apicidin or Depsipeptide; or
F. Hybrid molecules such as cyclic hydroxamic-acid-containing peptide (CHAP31) or CHAP50.

Component (ii) suitably comprises one or more of the inhibitors falling with the groups A-E above.

In particular, the combination of the invention comprises a HDAC inhibitory hydroxamic acid as component (ii), and this is suitably selected from SAHA or TSA.

Components of the combination of the invention are suitably provided in the form of pharmaceutical compositions, which contain further contain a pharmaceutically acceptable carrier or diluent.

These may be combined together in a single formulation, but preferably are packaged so that component (i) and component (ii) can be administered separately, for instance sequentially with component (ii) being used as a pretreatment, and component (i) being administered subsequently.

In this way, the mode of administration of the individual components may be different. For instance, component (i) generally needs to be applied directly to the site of a tumour, and therefore it is suitably formulated in a manner which facilitates this. For instance, component (i) may be in the form of topical compositions, infusions, suppositories, or compositions for inhalation or insufflation depending upon the position of the tumour being treated.

Component (ii) may be active systemically, and therefore, it may be administered using a wider range of routes, for example by oral or parenteral as well as those mentioned above for use in relation to component (i).

Where component (i) and component (ii) are admixed together, they will suitably be formulated for administration in a manner as described above in relation to component (i).

Depending upon the intended mode of administration, the compositions may therefore be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing.

Suitable pharmaceutically acceptable carriers or diluents would be understood in the art. They include pharmaceutically acceptable solid or liquid diluents.

In a particularly preferred embodiment, the compositions are pharmaceutical compositions in a form suitable for topical use, for example as creams, ointments, gels, or aqueous or oily solutions or suspensions. These may include the commonly known carriers, fillers and/or expedients, which are pharmaceutically acceptable.

Topical solutions or creams suitably contain an emulsifying agent for the protein complex together with a diluent or cream base.

The daily dose of the active compound varies and is dependant on the patient, the nature of the condition being treated etc. in accordance with normal clinical practice. As a general rule, from 2 to 200 mg/dose of component (i) is used for each administration.

Component (ii) is suitably administered in the normal therapeutic amount. For instance, a daily dose in the range, for example, 0.5 mg to 75 mg per kg body weight is received.

The invention further provides a combination as described above, for use in the treatment of proliferative disease, such as tumours and in particular cancer, as well as the treatment of pre-tumourous states such as those which may be caused through virus transformation.

Combinations as described above are particularly suitable for the treatment of tumours or other proliferative disease such as cancer, as well as papilloma and cells which have been transformed as a result of viral infection such as HPV and in particular HPV types (HPV 16 and 18) which are associated with cervical cancer.

In particular the combination may be used to treat malignant mucosal tumours or cancer such as bladder cancer, melanomas, cancers of internal organs, in particular, brain tumours, and any other condition, for instance cancers, where inhibition of angiogenesis is desirable.

Suitably, the combination is used in a sequential treatment in which component (ii) is administered first, and component (i) is administered subsequently, for example between 1 and 24 hours, suitably about 2-12 hours after component (ii). In this case, component (ii) may be administered systemically as described above, and component (i) is administered to the site of a tumour thereafter.

However, they components of the combination may be coadministered, in particular to the site of a tumour in a single formulation.

A further aspect of the invention provides a method of treating tumours or other proliferative disease such as cancer, or pre-tumourous conditions, which method comprises administering to a patient in need thereof a combination as described above. In particular, component (ii) of the combination is administered as a pre-treatment and component (i) is administered subsequently.

The applicants conducted a study to find if histone acetylation influenced the binding of HAMLET and if the interaction of HAMLET with chromatin was modified by acetylation.

Using the HDAC inhibitors TSA and SAHA, it was found that cells with acetylated chromatin were more sensitive to HAMLET, but this was not due to the interaction of HAMLET with the histone tail. During the course of this study, it was further noted however that the HDAC inhibitors significantly promoted the effect of HAMLET. A marked increase in the cell death response was noted.

It was found that cells which in particular had been pre-treated with HDAC inhibitors TSA and SAHA were more far more sensitive to HAMLET. This pre-treatment lead to an increase of cells showing fragmented DNA.

The effect was noted even when HAMLET was administered shortly (2 hours) after the HDAC inhibitor, which may indicate that the mechanism HAMLET was independent of protein (p21WAF1 and p27KIP1) neo-synthesis, commonly obtained after prolonged exposure to HDAC inhibitors. However, increased cell sensitivity persisted even after a prolonged use of HDAC inhibitors prior HAMLET treatment.

This indicates that HAMLET is a strong inducer of DNA fragmentation in the cancer cells, which can be enhanced to a higher than expected level by HDAC inhibitors.

Chromatin undergoes constant structural modifications, and this dynamic process is essential to control gene expression. In eukaryotic systems, the transcription process is tightly controlled, mainly by the access of transcription factors to the target DNA. The nucleosomes are basic structural elements of chromatin formed by 146 bp of DNA and two tetramers of H2A, H2B, H3 and H4, forming a histone octamer.

Posttranslational modifications of the histones influence the chromatin structure through phosphorylation, methylation, acetylation, ubiquitination, and sumoylation, forming the “histone code” {Drummond, 2005 Annu. Rev. Pharmacol. Toxicol. 45:495-528}.

Specifically, histone acetylation has been shown to open the chromatin to transcription factors while the deacetylated chromatin is compact with a decreased gene transcription.

The affinity of HAMLET for histones was confirmed, as HAMLET influenced the assembly of nucleosomes from histones and DNA in vitro. In addition, HAMLET interacted with preformed nucleosomes and high concentrations of HAMLET disrupted the chromatin. The in vivo effect of HAMLET was found to depend to some extent on the state of chromatin acetylation.

However, HAMLET itself was shown to decrease the acetylation of chromatin but when used in combination with HDAC inhibitors, which increased acetylation, produced a strongly enhanced the cell death response to HAMLET. The histone tail was not involved in the binding to HAMLET which was confirmed using mutant histones lacking the tail.

It appeared from studies conducted that HAMLET and HDAC inhibitors have an opposite effect on histone acetylation, and therefore the enhancement of the cell death effect is surprising. It seems possible that HAMLET does not act via the HAT/HDAC system, and that the mechanism by which HAMLET alters acetylation is different from the HDAC inhibitors.

This may relate to the difference in targets between these molecules. While HDAC interacts with the histone tail, HAMLET was shown to bind histones also in the absence of the histone tail.

HAMLET appears to be the first example of a substance that modifies chromatin acetylation without interacting with the histone tail where acetylation takes place. The decrease in acetylation is consistent with the formation of very tight aggregates between HAMLET and nucleosomes. In these large aggregates, the availability of the histone tails for HAT/HDAC may be reduced. The decrease in acetylation may thus be a question of accessibility rather than specific interference with specific enzymatic pathways.

It is possible that cells with open chromatin due to acetylation are susceptible to the effects of HAMLET, while cells with closed, deacetylated chromatin are more resistant to these effects, leading to the observed synergy.

A series of experiments were conducted to examine the effect of HDAC inhibitors on the cell death response to HAMLET. In this case, the HDAC inhibitors were applied initially and increased chromatin acetylation. Control experiments showed that protein acetylation increased within 3 hours. In one experiment, short pre-treatment intervals were used to avoid unspecific effect of the HDAC inhibitors through transcription and neo synthesis of proteins like p21WAF1 and P27KIP1. This was important, since HDAC inhibitors stimulate the acetylation of several proteins in addition to histones.

Thereafter, the DNA fragmentation response to HAMLET was correlated with the histone acetylation level, suggesting that HAMLET had an increased accessibility of the chromatin when histones were acetylated.

A marked effect on DNA fragmentation was observed with both the different HDAC inhibitors used. Interestingly, DNA fragmentation increased dramatically and occurred more rapidly after pretreatment of the cells with those inhibitors and the response to HAMLET was more rapid than DNA fragmentation in cells exposed to proapoptotic agents like staurosporine or etoposide.

The invention will now be particularly described by way of example with reference to the accompanying Figures in which:

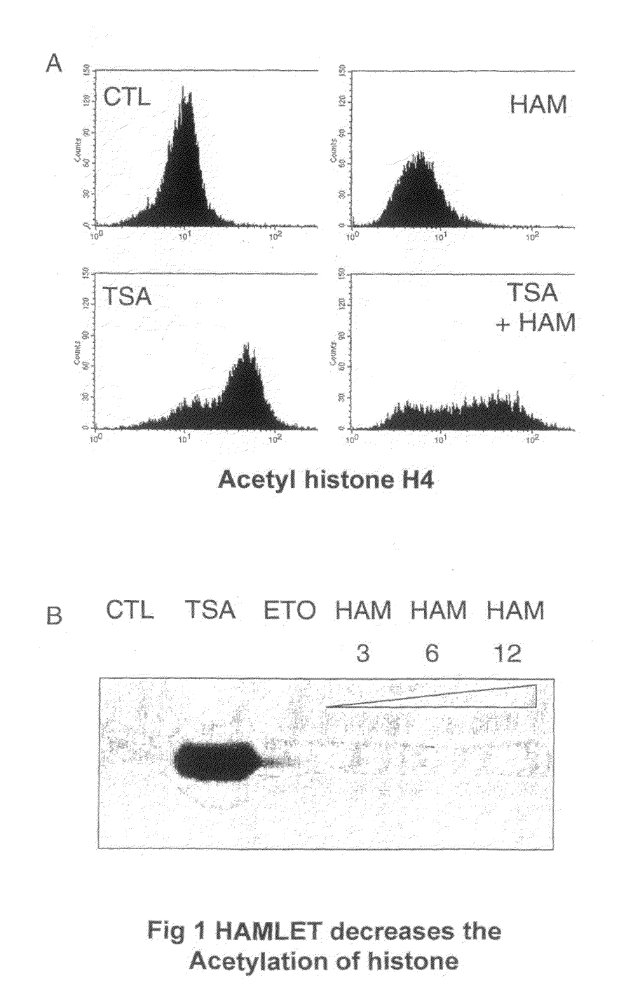

FIG. 1. Effect of HAMLET on histone acetylation in Jurkat cells.

A. Acetylation of Histone H4 was followed both by cytometry and NU-PAGE. In this experiment Jurkat cells were incubated 2H without (CTL and HAM) or with TSA 100 ng/ml (TSA and TSA+HAM), then HAMLET 12 μM was added in the medium in some conditions during 1H (HAM and TSA+HAM). B. Acetylation of Histone H4 was followed by NU-PAGE analysis. Jurkat cells (line 1) were treated 1H in presence of TSA 100 ng/ml (line 2), Etoposide 20 μM (line 3), HAMLET at 3 μM (line 4), 6 μM (line 5) and 12 μM (line 6).

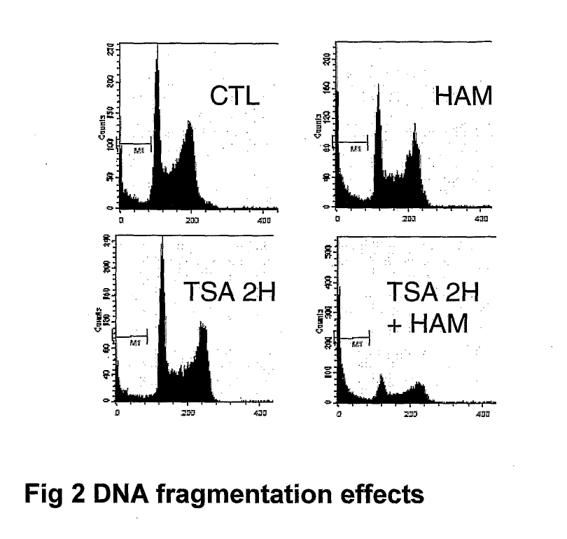

FIG. 2. HAMLET-induced rapid DNA fragmentation is enhanced by HDAC inhibitors. DNA fragmentation was followed both by flow cytometry. In this experiment Jurkat cells were incubated 2H without (CTL and HAM) or with TSA 100 ng/ml (TSA and TSA+HAM) or SAHA 2.5 μM (SAHA and SAHA+HAM), then HAMLET 12 μM was added in the medium in some conditions during 1H (HAM, TSA+HAM and SAHA+HAM). Following PI staining (cf Materials and methods), subG1 populations were quantified and presented as percent of the total population.

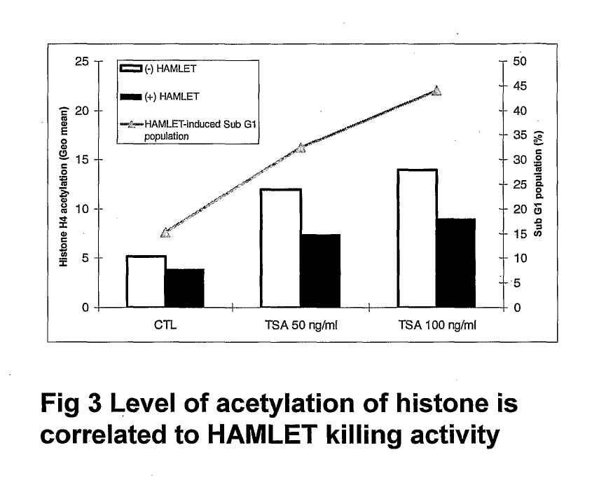

FIG. 3. Enhanced activity of HAMLET is correlated with the histone acetylation. In this experiment Jurkat cells were incubated 2H without (CTL) or with TSA 50 or 100 ng/ml (TSA50, TSA100), then HAMLET 12 μM was added in the medium in some conditions during 1H (Black Histograms). Then, acetylation of histone H4 in the different condition was quantified by cytometry and presented as the geometric mean of the population (Histograms, Left Scale). DNA fragmentation of the HAMLET-treated population was analysed with the sub G1 quantification and presented as percent of the total population (Line, Right Scale).

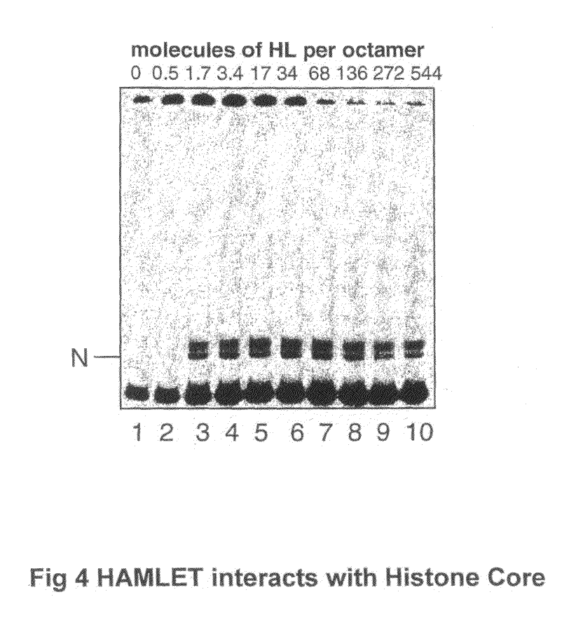

FIG. 4. Effects of HAMLET on a mixture of tailless histones and DNA. HAMLET was added to a mixture of radiolabelled 146 bp DNA fragments and tailless histones. In the absence of HAMLET, no nucleosomes are formed (lane 1). Addition of HAMLET first resulted in assembly of nucleosomes (lane 2). Nucleosome assembly on the 146 bp fragment results in one single nucleosome species, designated N. At higher concentrations of HAMLET, more nucleosomes were formed and HAMLET associated with them to form a second band in the gel. HAMLET also dissolved the unspecific histone-DNA aggregated seen in the wells.

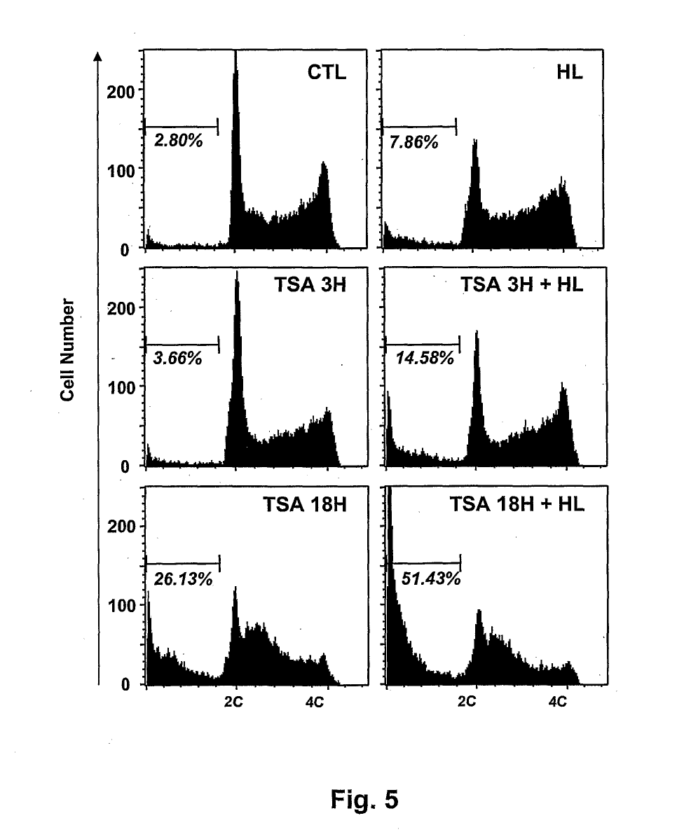

FIG. 5. HDAC inhibitors enhance HAMLET-induced DNA Fragmentation. DNA content was followed by flow cytometry. In this experiment Jurkat cells were first pretreated with or without TSA 100 ng/ml during 3H or 18H and then treated by HAMLET during 3H as described in the material and methods section. SubG1 population was quantified and presented in percent.

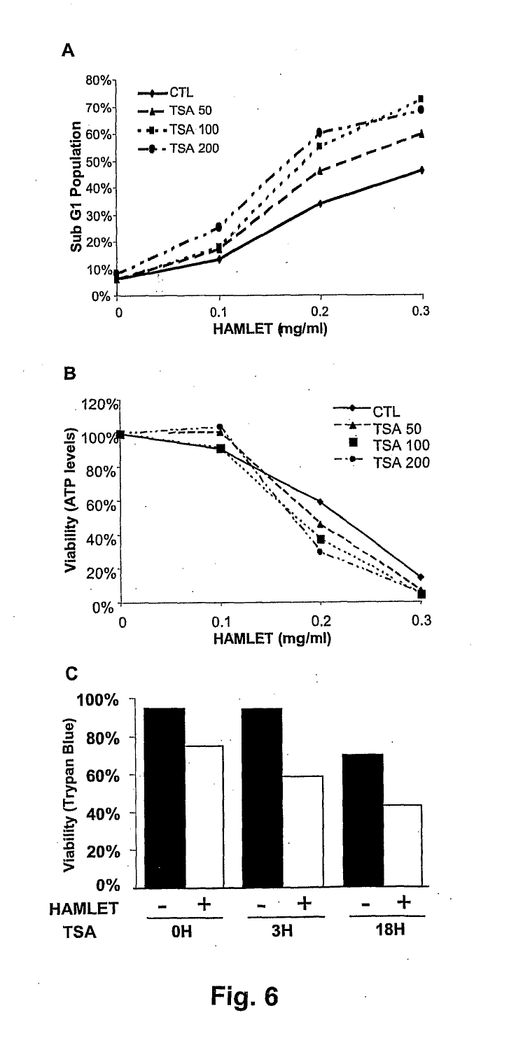

FIG. 6. HDAC inhibitors enhance HAMLET-induced cell death. A. In this experiment Jurkat cells were first pretreated with or without different concentrations of TSA 50, 100 or 200 ng/ml during 3H and then treated by increasing concentration of HAMLET during 3H. SubG1 population was quantified and presented in percent of the total population. B. Jurkat cells were first pretreated with or without different concentrations of TSA 50, 100 or 200 ng/ml during 3H and then treated by increasing concentration of HAMLET during 3H. ATP level were quantified as described in the Material and Method section. Results were given in comparison with the untreated population. C. Jurkat cells were first pretreated with or without TSA 100 ng/ml during 3 or 18H and then treated by HAMLET during 3H. Viability was assessed by trypan blue exclusion using the Vi-Cell XR apparatus.

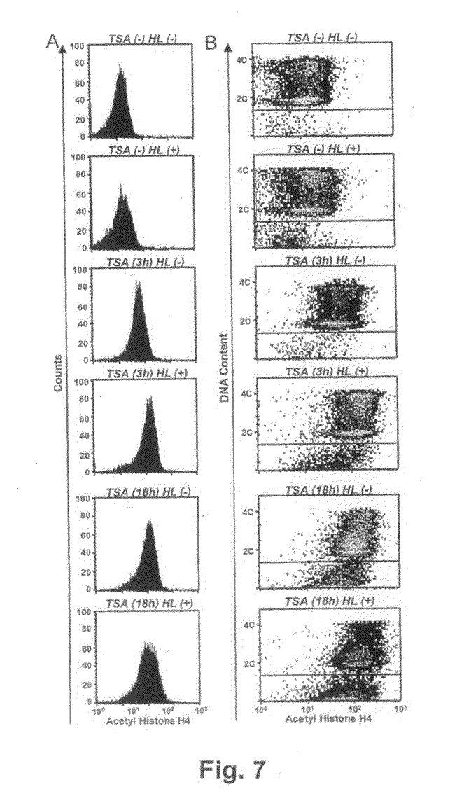

FIG. 7. Increased acetylation and cell death in cells treated both with HAMLET and TSA. A. In these experiments Jurkat cells were first pretreated with or without TSA 100 ng/ml during 3H or overnight (ON) and then treated by HAMLET during 3H. A. Acetylation levels were quantified by flow cytometry for the total population of cells by histogram plot. B. DNA content and acetylation of Histone H4 were followed by flow cytometry. Results were followed by density plot with in Y-axis the DNA content and in X-axis the histone acetylation.

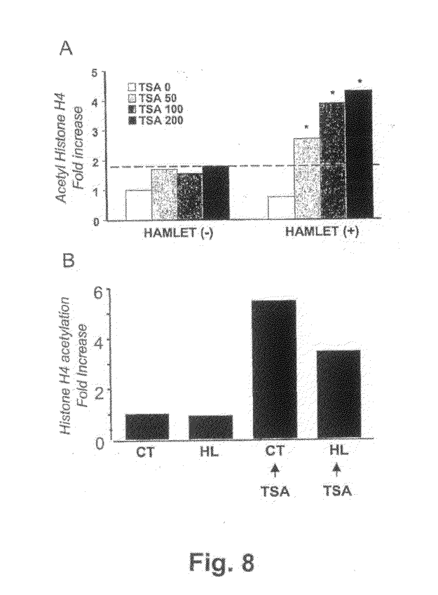

FIG. 8. Increased acetylation and cell death in cells treated both with HAMLET and TSA. A. Jurkat cells were first pretreated with or without different concentrations of TSA 50, 100 or 200 ng/ml during 3H and then treated by HAMLET during 3H. Results are presented in the fold of increase of the mean of the different “intact” populations versus the untreated “intact” population (* for p<0.001). B. In this experiment, Jurkat cells were first pretreated with or without HAMLET for 3H and then treated with or without 100 ng/ml during 3H (* for p<0.001).

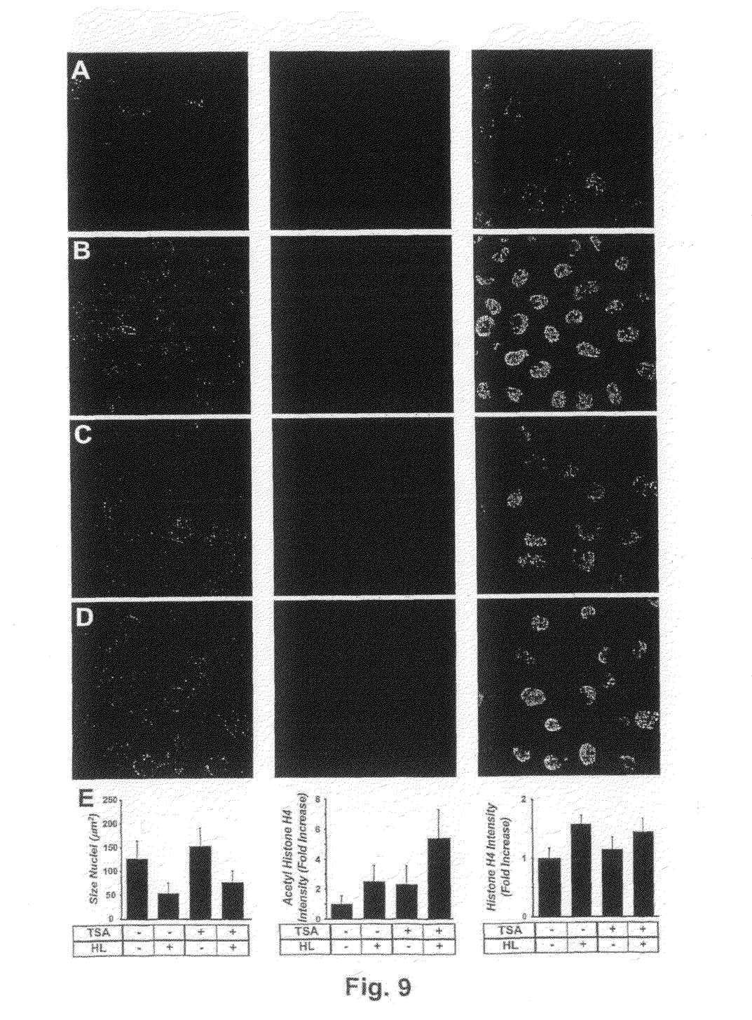

FIG. 9. Morphology of acetylated cells treated both with HAMLET and TSA. GFP-tagged histone H4 HeLa cells were untreated (A), or treated by HAMLET (B), or TSA 100 ng/ml (C), or pretreated by TSA and treated by HAMLET (D). First column represents the light transmission, the second column represents the acetylation of histone H4 and the third column represents the expression of histone H4 in the cells. E. Quantification on the size of the nuclei (μm²), the levels of histone H4 and acetyl histone H4 expression respectively. For the intensities of acetyl H4 and histone H4, results are presented in fold of increase of the control population. All results are expressed after counting of at least 30 cells for each experiment.

### EXAMPLE 1

### Effects of HAMLET on Histone Acetylation

### Materials and Methods

Reagents—HDAC inhibitors Trichostatin A (TSA) and Suberoylanilide hydroxamic acid (SAHA) were provided by Upstate (Dundee, UK) or Alexis (Lausen, Switzerland) and used at a concentration of 100 ng/ml and 2.5 μM respectively. ATP monitoring reagent (AMR) was purchased from Cambrex (Wokingham, U.K.) in the form of a ViaLight™ HS kit and was reconstituted according to the manufacturers guidelines.

Purification of α-Lactalbumin and Conversion to HAMLET—HAMLET is a folding variant of human α-lactalbumin stabilized by a C18:1 fatty acid cofactor. In this study, native α-lactalbumin was purified from human milk and converted to HAMLET on an oleic acid conditioned ion exchange matrix as previously described (Svensson et al., 2000).

Cell Culture—Jurkat (European Cell Culture Collection, no. 88042803 were cultured as described (Hakansson et al., 1995).

Histones—Native, folded histones were obtained from duck erythrocyte nuclei (Simon and Felsenfeld, 1979). Native or tailless Drosophila melanogaster histones were expressed in E. coli, purified and assembled into octamers (Hamiche et al., 2001). The fold and functional integrity of the histones were confirmed by nucleosome assembly on DNA (data not shown).

DNA—A 256-bp fragment containing a sea urchin 5S RNA gene (Simpson and Stafford, 1983) was gel-purified from an EcoR1 or Nci1 digest of plasmid pLV405-10 (Simpson et al., 1985). The DNA was end-labeled with [γ-³²P] ATP (Amersham Pharmacia biotech, UK).

Cell Cycle Analysis by cytometry—Cells were harvested, washed with PBS and then fixed in suspension in 75% cold ethanol at 4° C. for 2 hours at least. The samples were then centrifuged, washed with PBS, and treated with 0.25% triton X-100 for 10 minutes at room temperature. The cells were washed again, resuspended in 2.5 μg/ml of PI and 250 μg/ml Rnase A in PBS, and incubated at 4° C. overnight prior to measurement. Cellular fluorescence was measured using the Facscalibur flow cytometry (Becton Dickinson, San Jose, Calif.). The simple-cell populations were determinated on the basis of their fluorescence-intensity values FL2-A and FL2-W.

Acetyl histone H4 and Cell Cycle Analysis by cytometry—Cells were harvested, washed with PBS and then fixed in suspension in 75% cold ethanol at 4° C. for 2 hours at least. The samples were then centrifuged, washed with PBS, and treated with 0.25% triton X-100 for 10 minutes at room temperature. After addition of 3 ml of PBS and centrifugation, the cells were incubated for at least 30 min in swine serum 1% (DAKO, Solna, Sweden) in PBS. After addition of 3 ml of PBS and centrifugation, the cells were incubated for 3 hours at room temperature in the presence of rabbit polyclonal anti-body to human acetyl Histone H4 (Upstate), diluted 1/500 containing 1% BSA. Cells were then washed and incubated with a FITC-conjugated swine anti-rabbit antibody (DAKO, Solna, Sweden) diluted 1/20 in PBS containing 1% BSA for 2 h. The cells were washed again, resuspended in 2.5 μg/ml of PI and 250 μg/ml Rnase A in PBS, and incubated at 4° C. overnight prior to measurement. The control was prepared identically as described above, except the presence of the histone antibody. Cellular fluorescence was measured using the Facscalibur flow cytometry (Becton Dickinson, San Jose, Calif.). The simple-cell populations were determined on the basis of their fluorescence-intensity values FL2-A and FL2-W. Then red and green emissions from each cell were separated and quantified using the standard optics of the cytometer.

Acetyl histone H4 by confocal microscopy—Hela Cells were grown in Lab-Tek Chamber slides. Cells were washed twice in PBS, and then fixed 10 minutes in PBS formaldehyde 4%. The samples were then centrifuged, washed with PBS, and treated with 0.1% triton X-100 for 2 minutes at room temperature. After washes, cells were incubated for at least 30 min in swine serum 1% (DAKO, Solna, Sweden) in PBS. Then cells were incubated for 3 hours at room temperature in the presence of rabbit polyclonal antibody to human acetyl Histone H4 (Upstate), diluted 1/500 containing 1% BSA. Cells were then washed and incubated with an Alexa633-conjugated goat anti-rabbit antibody (Invitrogen) diluted 1/200 in PBS containing 1% BSA for 2 h. The cells were washed 3×5 min. in PBS prior to evaluation in an LSM 510 META confocal microscope (Carl Zeiss, Germany) with a 63× objective. The frequency of each chromatin patterns is shown in fold of control cells staining after counting a minimum of 30 cells in each experiment.

Immunoblot—JURKAT cells were washed twice in ice-cold PBS and lysed in lysis buffer [20 mM Tris-Cl (pH 7.5), 100 mM NaCl, 5 mM MgCl₂, 0.5% Nonidet P-40] supplemented with protease inhibitors (Roche Applied Science). After 30 min at 4° C. under continuous agitation, lysates were submitted to H₂SO₄ (0.4N) during 1 h before centrifugation at 12,000 g for 15 min at 4° C. 50 μg of protein extracts were separated on SDS-PAGE, and were electrotransferred onto a polyvinylidene fluoride membrane (Immobilon-P, Millipore). The membrane was incubated overnight at 4° C. anti-acetyl Histone H4 ( 1/2000) (Upstate). Horseradish peroxidase-conjugated goat anti-rabbit antibody (1:10,000) (Dako A/S Denmark) was then applied for 1 hour at room temperature. Immunoreactive bands were revealed by enhanced chemiluminescence (ECL, Amersham).

DNA Fragmentation—High molecular weight DNA fragments were detected by field-inversion gel electrophoresis (FIGE) as described (Zhivotovsky 1993 207 163). Briefly, cells (2×10⁶) were embedded in low melting point agarose gel treated by proteinase K. Samples were run by electrophoresis at 180 V in 1% agarose gels in 0.53 TBE (45 mM Tris, 1.25 mM EDTA, 45 mM boric acid, pH 8.0), at 12° C., with the ramping rate changing from 0.8 s to 30 s for 24 h, using a forward to reverse ratio of 3:1. Quantification of High molecular fragmented DNA bands was performed using imageJ software.

### Effects of HAMLET on Histone Acetylation

The level of chromatin acetylation in Jurkat cells was quantified by flow cytometry, using specific antibodies to acetylated histone H4 (FIG. 1). To increase acetylation, the cells were treated with the HDAC inhibitor TSA (FIG. 1C, FIG. 1E line 3). The maximum maximal effect was seen after two hours. The increase in acetylation was confirmed by Western Blot analysis of nuclear extracts from the TSA treated cells.

Low concentrations of HAMLET on Jurkat cells caused a decrease in histone H4 acetylation compared to the medium control (FIG. 1B, Fig. E line 2) (FIG. 1A, FIG. 1E line 1). This decrease in acetylation was confirmed by Western blot analysis.

The results suggested that HAMLET at low concentrations may act as an acetylation inhibitor or that HAMLET may kill cells with acetylated chromatin leaving only the ones with low acetylation in Jurkat cells.

The combined effect of HAMLET and TSA on H4 acetylation is shown in FIG. 1D, FIG. 1E line 4) (FIG. 1C, FIG. 1E line 3). The reduction is acetylation by HAMLET was greater in the TSA pre-treated cells than after HAMLET treatment alone.

### EXAMPLE 2

### HDAC Inhibitors Increase DNA Fragmentation in Response to HAMLET

The effects of HAMLET on DNA fragmentation were compared between untreated cells and those pre-treated with TSA to increase acetylation. Sub G0/G1 DNA fragmentation was quantified by flow cytometry using the methodology as described in Example 1.

HAMLET was shown to induce DNA fragmentation in Jurkat cells after 1 hour (FIG. 4A) but TSA treatment did not stimulate DNA fragmentation. TSA pre-treatment increased the susceptibility to HAMLET, however, and enhanced HAMLET-induced DNA fragmentation.

The susceptibility of the cells to HAMLET was directly related to the TSA concentration, suggesting that HDAC inhibitors increase tumour cell death induced by HAMLET (FIG. 2).

The effect of HDAC inhibitors was examined further by comparing TSA to SAHA, and alternative HDAC inhibitor. Jurkat cells were pre-treated with TSA (100 ng/ml) or SAHA (2.5 μM) for 2 hours, and exposed to HAMLET for 1 hour and sub G0/G1 DNA fragmentation was quantified by flow cytometry (FIG. 3B). DNA fragmentation in response to HAMLET was enhanced by SAHA (FIG. 3D), but the substance itself did not trigger DNA fragmentation (FIG. 3C).

In addition, non confluent A459 cells were treated with or without TSA 100 ng/ml overnight. Then HAMLET was added for one hour at 30 μM. As before, DNA fragmentation was followed both by flow cytometry. Following PI staining (cf. Materials and methods), subG1 populations were quantified and presented as percent of the total population.

Again, it appeared that long exposure to TSA significantly increased cell death (data not shown).

### EXAMPLE 3

### HAMLET Interacts with Histone Core

Histones exert many of their effects through the 20 amino acid histone tail which is more accessible than other histone domains and may be modified by various stimuli. The binding of HAMLET was compared between wild-type histones and mutants lacking the histone tail.

Histones—Native, folded histones were obtained from duck erythrocyte nuclei. Native or tailless Drosophila melanogaster histones were expressed in E. coli, purified and assembled into octamers. The fold and functional integrity of the histones were confirmed by nucleosome assembly on DNA.

DNA—A 256-bp fragment containing a sea urchin 5S RNA gene (Simpson and Stafford, 1983) was gel-purified from an EcoR1 or Nci1 digest of plasmid pLV405-10 (Simpson et al., 1985). The DNA was end-labeled with [γ-³²P] ATP (Amersham Pharmacia Biotech, UK).

Chromatin assembly—To generate nucleosomes, histone octamers (recombinant or purified from cells) were assembled on linear DNA according to the “salt jump” method (Stein, 1979). ³²P-labeled 256 bp fragments and carrier DNA (supercoiled plasmid DNA, final DNA concentration 200 μg/ml) were mixed with histones (histone:DNA weight ratio 0.4-0.6) in 2 M NaCl, 10 mM Tris-HCl (pH 7.5). The mixture was incubated for 10 min at 37° C., diluted to 0.5 M NaCl, incubated at the same temperature for 30 min, and dialyzed at 4° C. against 10 mM Tris-HCl (pH 7.5) and 1 mM EDTA for 2 h. For experiments involving SYBR green staining of the gels, the carrier plasmid DNA was substituted with non-radioactive 256 bp DNA fragments. The chromatin was stored at 4° C.

### EXAMPLE 4

### HDAC Inhibitors Enhance HAMLET-Induced Cell Death

To examine if HDAC inhibitors might influence the effects of HAMLET on the viability of tumour cells, Jurkat cells were pretreated with HDAC inhibitors TSA 3 hours or 18 h and then exposed to HAMLET (FIG. 5). The cellular response to the combined treatment was compared to the response to TSA alone (3 h or 18 h) and to HAMLET (3 h). Cells in medium were used as controls. The cellular response was quantified by flow cytometry and the subG1 population was used as a measure of chromatin fragmentation and cell death (FIGS. 5 and 6A). In parallel, cell viability was assessed by ATP levels (FIG. 6B) and trypan blue exclusion (FIG. 6C).

Short term exposure to the HDAC inhibitors (TSA, 3 h) did not cause detectable changes in DNA fragmentation compared to the control (3.66 vs. 2.80%), but 18 hours TSA exposure caused the expected increase in subG1 population (26.13 vs. 2.80). For both TSA alone and HAMLET alone, the increase in the subG1 population was accompanied by a loss of cell viability (FIG. 6B and FIG. 6C). Gradual increased concentrations of HAMLET induced a decrease in ATP levels in the cells (9.2% vs. 41.17% vs. 85.71% decreases after 3 h exposure with HAMLET at 0.1 mg/ml, 0.2 mg/ml, and 0.3 mg/ml, respectively) (FIG. 6B). By trypan blue exclusion the loss of viability was 19.7% after HAMLET treatment 3 h in comparison with control. The loss of viability after TSA treatment was detectable only after 18 h exposure with a decrease of 24.9% in comparison with control (FIG. 6C).

TSA pretreatment enhanced cell death in response to HAMLET (FIG. 5, FIG. 6). The subG1 population increased from 7.68% in TSA treated cells (3 h) to 14.58% in TSA+HAMLET treated cells. The accumulation in subG1 was further enhanced by 18 hours exposure with TSA (51.43% in TSA+HAMLET vs. 26.13% in TSA treated vs. 14.58% in HAMLET). The effect was concentration dependant as shown by the increase from 50 to 200 ng/ml of TSA (FIG. 6A). The increase of subG1 population was correlated to a gradual decrease in viability of Jurkat cells exposed with increased periods or doses of TSA pretreatment (FIG. 6B and FIG. 6C). Pretreatment with gradual increased concentrations of TSA before HAMLET treatment potentiate the decrease in ATP levels observed by HAMLET alone or TSA alone in the cells (3 h pretreatment with TSA at 50 ng/ml, 100 ng/ml, and 200 ng/ml followed by HAMLET treatment (0.2 mg/ml) decreased the ATP levels by 54.38%, 63.03%, and 70.61% respectively vs. 41.17% for HAMLET alone in comparison with control cells) (FIG. 6B). By trypan blue exclusion the loss of viability was enhanced after increased time pretreatment with TSA followed by HAMLET treatment 3 h (36% after 3 h TSA pretreatment and HAMLET in comparison with control and 51.7% after 18 h TSA pretreatment and HAMLET vs. HAMLET alone (19.7%) vs. TSA alone 18 h (24.9%).

The results showed that HDAC inhibitors increased the lethality of HAMLET for tumour cells.

### EXAMPLE 5

### Effects on Histone H4 Acetylation

The inhibition of HDAC causes an accumulation of acetylated histones. The level of acetyl histone H4 in Jurkat cells was quantified by flow cytometry after staining with specific antibodies. The cells were counterstained with propidium iodide to visualize the change in chromatin levels. Results are presented as density plot diagrams representing the DNA content on the Y-axis between four populations of cells (Sub1, G1, S, and G2), and the degree of acetylation of Histone H4 on the X-axis. Four populations of cells (Sub1, G1, S, and G2) could be distinguished on the Y-Axis.

TSA caused a time dependent increase in histone acetylation (FIG. 7). Jurkat cells were exposed to TSA for 3 hours or 18 h and the increased of acetylation was quantified as described above. In contrast, HAMLET alone did not change the degree of acetylation, but in combination with TSA, a significant increase was observed. The increase of acetylation was more rapid and higher in cells exposed to TSA and HAMLET than after exposure to TSA alone.

Analysis of the acetylation of the different cell populations revealed that HAMLET triggers an increase in the acetylation of histone H4 and that acetylation of histone H4 was always less important in all the subG1 population in comparison with the resting or “intact” populations independently of the treatment performed on the cells (FIG. 7). Moreover, results showed by comparing the total population of cells versus the “intact cells”, that the accumulation of the subG1 population could interfered with the mean of acetylation of the total population (FIG. 7).

Then, by removing the subG1 population from the analysis, an increased acetylation of histone H4 was shown in the resting “intact” cell populations treated with both TSA and HAMLET compared to those treated with TSA alone (FIG. 8A). After 3 h treatment with TSA, HAMLET induced a 2 fold increase in the acetylation of histone. The acetylation degree induced by HAMLET and TSA was higher after 3 h than the degree of acetylation induced by an over night exposure with TSA (FIG. 8A). Moreover, HAMLET in presence of TSA was still able to promote the acetylation of the chromatin even after overnight exposure to TSA (FIG. 8A). Similar results were found with another HDAC inhibitor SAHA when it was combined with HAMLET treatment (data not shown). Interestingly, by subjecting cells to increased concentration of TSA without HAMLET, the rate of acetylation was still the same in the JURKAT cells with an increase of 2 fold of the control. When HAMLET was added, an increase of the acetylation of histone H4 was shown that was TSA dose-dependent with an increased of acetylation from 2.7 to 4.3 fold of the control (FIG. 8A).

These results showed that HAMLET at high concentrations in combination with HDAC inhibitors promotes an increase of acetylation in the cell.

It was then analyzed how important was the HDAC inhibitor pretreatment of the Jurkat lymphoid cells prior to HAMLET treatment. It was then compared whether HDAC inhibitors could be used before or after HAMLET. In a first experiment the cells were pre-treated with HDAC inhibitors and treated with HAMLET. As previously described, an enhanced acetylation was found in the combined procedure (FIG. 8A). Then, the cells were first treated with HAMLET and then TSA was added. Interestingly the level of acetylation was then decreased (FIG. 8B), showing that the cells have to be sequentially pretreated by HDAC inhibitors and treated by HAMLET to have an enhanced acetylation of acetyl histone H4.

These results showed that the sequence of treatment is very important for getting synergistic effect in term of acetylation in lymphoid cells.

### EXAMPLE 6

### HAMLET Effects on TSA-Induced Chromatin Modification

The effect of HAMLET and TSA on the acetylation of Histone H4 was further examined by confocal microscopy of adherent HeLa cells expressing GFP-tagged Histone H4. Control cells showed nuclei with some nucleoli as shown by the H4-GFP staining. The acetylation level of histone H4 in the control cells was very weak (FIG. 9A). HAMLET treated cells arbors a different shape with a marked decrease in the size of the nuclei and an increase of the intensity of the GFP staining. Interestingly, in epithelial cells HAMLET did induce an increase in the acetylation of Histone H4 (FIGS. 9B and E). TSA treated cells showed a slight increase in the size of the nuclei and in the staining of the GFP, and as expected an increase in the staining of the acetylation of histone H4 (FIGS. 9C and E). Even if the increase of acetylation was the same between HAMLET and TSA at this time in these cells, the shape of the nuclei in the two different conditions showed completely opposite kind of responses (FIGS. 9B and C and E). Moreover, when cells were pretreated by TSA and treated by HAMLET, a HAMLET-like response was found with a decrease in the size of the nuclei and an increase of the GFP staining. In this condition, the level of acetylation was synergistically enhanced in the combined treatment (FIGS. 9D and E) confirming on an epithelial cell type the synergistic effect found of Jurkat lymphoid cells by cytometry analysis.

These results showed that HAMLET in combination with HDAC inhibitors also promotes an increase of acetylation in epithelial cell. Moreover, by looking at the shape of nuclei, it was concluded that the HAMLET-induced increase of acetylation was not correlated to an HDAC inhibitor response, but rather to a stress of the nuclei with shrinking deformation.

1. A combination of component (i) which is HAMLET or a biologically active modification thereof, or a biologically active fragment of either of these, and component (ii) which is a histone deacetylase (HDAC) inhibitor.

2. A combination according to claim 1 wherein component (i) is HAMLET.

3. A combination according to claim 1 wherein component (ii) is an HDAC inhibitory carboxylic acid of low molecular weight, an HDAC inhibitory hydroxamic acid, an HDAC inhibitory benzamide, and HDAC inhibitory epoxyketone, and HDAC inhibitory cyclic peptide or a hybrid or mixture of any of these.

4. A combination according to claim 3 wherein component (ii) is selected from Trichostatin A (TSA) or suberoylanilide hydroxamic acid (SAHA).

5. A combination according to claim 1 which is packaged so that component (i) and component (ii) can be administered separately.

6. A combination according to claim 1 wherein components (i) and (ii) are mixed together.

7. A combination according to claim 1 for use in the treatment of tumours or other proliferative disease or pre-tumourous states.

8. A combination according to claim 7 which is packaged so that component (i) and component (ii) can be administered separately, for use in a sequential treatment in which component (ii) is administered first, and component (i) is administered subsequently.

9. A combination according to claim 8 wherein component (i) is administered between 1 and 24 hours after component (ii).

10. A combination according to claim 7 for use in a treatment wherein component (i) and component (ii) are coadministered.

11. A combination according to claim 10 wherein component (i) and component (ii) are coadministered to the site of a tumour in a single formulation.

12. A combination according to claim 10 wherein component (i) is administered to the site of a tumour and component (ii) is administered systemically.

13. A method of treating tumours or other proliferative disease or pre-tumourous states which method comprising administering to a patient in need thereof a combination according to claim 1.

14. A method according to claim 13 wherein the proliferative disease is a tumour.

15. A method according to claim 13 wherein component (ii) of the combination is administered as a pre-treatment and component (i) is administered subsequently.
